(19) United States
(12) Patent Application Publication
(10) Pub. No.: US 2026/0309699 A1
(43) Pub. Date: Oct. 8, 2026

(54) PREDICTION OF IMMUNOSTIMULATORY POTENTIAL OF AAV IN A HUMAN PRECLINICAL MODEL

(71) Applicant: The Board of Trustees of the Leland Stanford Junior University, Stanford, CA (US)

(72) Inventors: Bradley Hamilton, Palo Alto, CA (US); John F. Wright, Redwood City, CA (US)

(21) Appl. No.: 19/476,121

(22) PCT Filed: Apr. 23, 2024
(86) PCT No.: PCT/US2024/025821

§ 371 Date: Oct. 16, 2025

Related U.S. Application Data
(60) Provisional application No. 63/461,533, filed on Apr. 24, 2023.

(51) Int. Cl. G01N 33/50

(52) U.S. Cl. CPC G01N 33/5047; G01N 2333/015

(57) ABSTRACT

Compositions and methods are provided for predicting the strength of an immune response that is elicited in humans following administration of a specific rAAV vector. The methods utilize antibody-opsonization of rAAV to enhance assays to reveal and quantify immune responses in human PBMC based assays.

### CROSS-REFERENCE TO RELATED APPLICATIONS

This application is a U.S. National Stage of PCT Application No. PCT/US2024/025821, filed Apr. 23, 2024, which application claims the benefit of U.S. Provisional Patent Application No. 63/461,533, filed Apr. 24, 2023, the disclosures of which applications are incorporated herein by reference in their entirety.

### GOVERNMENT SUPPORT RESEARCH

This invention was made with Government support under contract NS120137 awarded by the National Institutes of Health. The Government has certain rights in the invention.

### BACKGROUND

Recombinant adeno-associated virus (rAAV) gene therapy (GT) vectors are transformative FDA approved treatments for Leber congenital amaurosis, spinal muscular atrophy, and hemophilia B. These are early examples of what could become a new treatment paradigm for broad ranging genetic indications. Alongside these clear successes, several investigational stage rAAVs have encountered significant barriers to product development due to immunotoxicities, particularly when high doses are required, as with Duchenne muscular dystrophy. The inability to adequately model deleterious immune responses to rAAV has emerged as a critical shortcoming. One of the most concerning is innate immune responses, such as those initiated through toll-like receptor (TLR) activation and complement activation.

Most of the severe adverse events (SAEs) that have occurred in the clinic, including 8 level 5 SAEs (deaths) deemed to be investigational product-related reported in the last two years in four independent trials for different indications, were not predicted because they were inadequately modeled. Current methods utilize discordant rodent models requiring extensive modification, with 12 TLRs and 14 interferon alpha (IFNα) subtypes, compared to 10 and 13 in humans, respectively. Recent reports suggest the sensitivity of TLR9, a key player in host response to rAAV, is dramatically enhanced in great apes (including humans) over lower mammals.

Cell line based models are also used, which can sufficiently measure TLR9 activation by oligodeoxynucleotides (ODNs) but are non-physiological and poorly stimulated by rAAV. Ideally, systems employing primary human cells, including peripheral blood mononuclear cells (PBMCs), would be used to predict immune responses to rAAV. However, PBMCs are highly variable because they are obtained from human donors, and to date have been insensitive to potentially significant differences between related rAAVs. There is therefore an acute and recognized unmet need for a physiological and sensitive human model to accurately predict adverse immune responses preclinically.

The ability to accurately predict adverse immune responses in a preclinical model would permit evaluation and selection of optimal lead rAAV candidates to support rAAV based gene therapy (GT) indications where minimizing immune response to the product is desired; would permit evaluation and selection of lead rAAV candidates to support rAAV based vaccine product development where maximizing immune response to the product is desired; would provide validation of lot-to-lot consistency, safety and efficacy during GMP rAAV vector production; or would provide a companion diagnostic tool and basis for human subject inclusion in clinical trials using investigational rAAV products and patients for licensed rAAV products.

Recombinant AAV vectors are discussed by in Wright (2020) Molecular Therapy 28(3):701-703; and Hamilton and Wright (2021) Front. Immunol. 12:675897.

The present disclosure provides tools and methods for this analysis.

### SUMMARY

Compositions and methods are provided for predicting the strength of an immune response that is elicited in humans following administration of an rAAV vector of interest. In some embodiments, predictive distinctions are made between related rAAV vectors, e.g. vectors that have a high degree of sequence similarity. The methods of the invention utilize antibody-opsonization of rAAV to enhance assays that exploit fundamental physiological processes, to reveal and quantify immune responses in human PBMC based assays.

In some embodiments, improved assays are provided for detecting the presence and strength of an AAV-specific cell-mediated immune response in an individual. In the methods of the invention, a sample comprising immune cells, e.g. dendritic cells, monocytes, T cells, Bc cells, NK cells, etc., usually a blood sample or derivative thereof including, without limitation, a peripheral blood mononuclear cell sample, is contacted with an effective dose of a test AAV composition in the presence of an effective amount of AAV-specific opsonizing antibodies. Opsonizing antibodies of interest specifically bind to AAV, and comprise an Fc region sequence that binds to Fc receptors present on the immune cells. The sample is incubated for a period of time sufficient to allow for uptake of the opsonized AAV, activation of responsive immune cells; and release of IFN-α or other cytokine or immune effector molecule(s) by the responsive cells. The level of IFN-α or other cytokine or immune effector molecule(s) produced by the responsive immune cells is indicative of the level of cell mediated responsiveness to the AAV composition. Use of an opsonizing antibody in the assay is shown herein to increase the sensitivity of the assay.

In some embodiments the immune cells are PBMC. The immune cells may be isolated from an individual, for example for use in determining the suitability of an AAV composition for administration to the individual, e.g. in a gene therapy application. In other embodiments the immune cells are isolated from an individual for AAV evaluation purposes, and may be combined in a panel of cells from a range of individuals. In other embodiments the immune cells are isolated from a non-human source, e.g. for pre-clinical testing of an AAV composition.

In some embodiments the immune effector protein is a cytokine or a non-cytokine effector protein, including without limitation IFN-α, and pro-inflammatory cytokines such as IL-1, IL-2, IL-6, IL-12, IL-17, IL-18, IFN-γ, and TNF-α. The presence or level of an immune effector protein of interest may be determined at the level of the protein itself, or to the extent to which a gene encoding the protein is expressed. Conveniently, assays known in the art for measuring cytokine release may find use in the methods of the invention, for example with antibody, nucleic acid, or protein-based readouts, including without limitation western blot, flow cytometry, PCR, ELISA, Luminex, ELISpot, cyTOF, and tetramer assays and the like.

In some embodiments the AAV-specific opsonizing antibody is polyclonal, e.g. antibodies from AAV capsid-seropositive blood, plasma or sera obtained from human subjects subsequent to rAAV administration. In some embodiments the AAV-specific opsonizing antibody comprises AAV capsid-seropositive blood, plasma or taken from research animals subsequent to rAAV administration. In some embodiments the AAV-specific opsonizing antibody comprises AAV capsid-specific monoclonal antibodies, which may be engineered or selected to have an Fc region that can bind to human Fc receptors to effect human Fc effector functions. Alternatively a monoclonal antibody comprises an Fc region that can bind to non human Fc receptors, in order to effect Fc effector functions in a non-human animal model. Monoclonal antibodies may be chimeric, for example, a human Fc domain combined with an AAV capsid specific mouse monoclonal (Fab)₂ domain; humanized, e.g. from a mouse antibody; or fully human.

Test antigens of interest for the assay include any AAV composition. Of interest are recombinant AAV that are used, or evaluated for use, in gene therapy treatment. Preferred compositions comprise an AAV genome associated with capsid proteins. One or a plurality, e.g. a panel, of test AAV compositions may be assayed. A feature of the present methods is the ability to distinguish between rAAV that differ only in genomic sequences.

In some embodiments an individual is tested for responsiveness to a candidate rAAV composition by the methods disclosed herein prior to treatment of an individual with the rAAV. An individual that is determined to have a low level of responsiveness may be treated with the candidate rAAV. An individual that has a high level of responsiveness to the rAAV may be treated with an alternative rAAV, or with a non-AAV therapeutic modality.

The present invention further provides kits comprising reagents and compartments required to perform the assay. Generally, the kit further comprises a set of instructions. The assay may also be automated or semi-automated and the automated aspects may be controlled by computer software.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1: AAV GT increases serum neutralization of AAV2-CAG>GFP on Hela cells. AAV2 carrying a GFP transgene driven by the CAG promoter was incubated with serial dilutions of either serum from a human patient yet to receive AAV treatment, serum from that same patient after receiving AAV treatment, or a mouse monoclonal antibody (A20) for one hour. Then, AAVs were overlaid on Hela cells cultured to 90% confluent monolayer in each well of a black opaque 96 well tissue culture plate, in triplicate. Four days later, the fluorescence intensity of each well was measured with a plate reader.

FIG. 2: rAAV2-GFP elicits 19.76 pg/mL more INFα1 than rAAV2-empty capsids from hPBMCs. Human PBMCs were isolated from three random healthy donors by ficoll gradient centrifugation and plated in a 96 well U-bottom tissue culture plate. Immediately after plating, either AAV2-empty capsid, AAV2-GFP, ODN2216, or ODN2216 with methylated CpGs were overlaid on PBMCs, in triplicate. 24 hours later, the supernatant was removed, technical replicated were pooled, and ELISA performed to measure INFα1 concentrations.

FIG. 3: rAAV2-GFP elicits 727.27 pg/mL more INFα1 than rAAV2-empty capsids, after post-GT serum incubation. Human PBMCs were isolated from three random healthy donors by ficoll gradient centrifugation and plated in a 96 well U-bottom tissue culture plate. Immediately after plating, either AAV2-empty capsid pre-treated for one hour with serum taken from an AAV therapy recipient, AAV2-GFP pre-treated for one hour with serum taken from an AAV therapy recipient, ODN2216, or ODN2216 with methylated CpGs were overlain on PBMCs, in triplicate. 24 hours later, the supernatant was removed, technical replicated were pooled, and ELISA performed to measure INFα1 concentrations.

FIG. 4: FcR antibodies blunt serum-mediated signal amplification. Human PBMCs were isolated from six random healthy donors by ficoll gradient centrifugation and plated in a 96 well U-bottom tissue culture plate. To one set of wells anti-human Fc blocking antibody was added. All cells were then overlain with either AAV2-empty capsid, AAV-GFP, or AAV2-GFP pre-treated for one hour with serum taken from an AAV therapy recipient, in triplicate. 24 hours later, the supernatant was removed, and ELISA performed to measure IL-6 concentrations.

FIG. 5: Incubation with AAV converted serum increases CpG-dependent CTL activation. Human PBMCs were isolated from three random healthy donors by ficoll gradient centrifugation and plated in a 96 well U-bottom tissue culture plate. Immediately after plating, either AAV2-empty capsid, AAV2-empty capsid pre-treated for one hour with serum taken from an AAV therapy recipient, AAV2-GFP, or AAV2-GFP pre-treated for one hour with serum taken from an AAV therapy recipient were overlain on PBMCs, in triplicate. 72 hours later, cells were stained and analyzed by fluorescence activated cell sorting (FACS) using a flow cytometer. CD3+/CD8+ cells were interrogated for the presence of CD25 and CD65 activation markers.

FIG. 6: Incubation with anti-AAV mAb (A20) does not alter hPBMC elicited IFNα1. Human PBMCs were isolated from three random healthy donors by ficoll gradient centrifugation and plated in a 96 well U-bottom tissue culture plate. Immediately after plating, either AAV2-GFP, AAV2-GFP pre-treated for one hour with serum taken from an AAV therapy recipient, ODN2216, or ODN2216 with methylated CpGs were overlain on PBMCs, in triplicate. 24 hours later, the supernatant was removed, technical replicated were pooled, and ELISA performed to measure INFa1 concentrations.

FIG. 7: Incubation with anti-AAV mAb (A20) does not alter mPBMC elicited IL2α. Murine PBMCs were isolated by ficoll gradient centrifugation after extraction from 6 female and 6 male mice, pooled by sex, and plated in a 96 well U-bottom tissue culture plate. Immediately after plating, cells were overlain with either AAV2-empty capsid, AAV2 CMV>null vector, AAV2>CAG>GFP, PMA positive control, or these same recombinant AAVs pre-treated for one hour with serial dilutions of murine anti-AAV2 (A20), in triplicate. 24 hours later, the supernatant was removed, technical replicated were pooled, and Luminex performed in duplicate. mIL2Rα results are shown.

FIG. 8: hPBMCs from random donors can be unresponsive to CpGs. Human PBMCs were isolated from three random healthy donors by ficoll gradient centrifugation and plated in a 96 well U-bottom tissue culture plate. Immediately after plating, either ODN2216, or ODN2216 with methylated CpGs were overlain on PBMCs, in triplicate. 24 hours later, the supernatant was removed, technical replicated were pooled, and ELISA performed to measure INFα1 concentrations.

FIG. 9: Related AAV vectors cannot be differentiated by genomic immunogenicity. Human PBMCs were isolated by ficoll gradient centrifugation after extraction from three random donors and plated in a 96 well U-bottom tissue culture plate. Immediately after plating, cells were overlain with either AAV2-empty capsid, AAV2 CMV>null vector, or AAV2>CAG>GFP, in triplicate. 24 hours later, the supernatant was removed, and ELISA performed to measure INFα1 concentrations.

FIG. 10: hA20 opsonization distinguishes closely related AAVs by genomic immunogenicity. Human PBMCs were isolated by ficoll gradient centrifugation after extraction from three random donors and plated in a 96 well U-bottom tissue culture plate. Immediately after plating, cells were overlain with either AAV2-empty capsid, AAV2 CMV>null vector, or AAV2>CAG>GFP all pre-treated for one hour with chimeric anti-AAV2 mAb hA20 (murine Fab fused to human Fc), in triplicate. 24 hours later, the supernatant was removed, and ELISA performed to measure INFa1 concentrations.

FIG. 11: hA20 preincubation is elucidating key innate immune responses to AAV. Human PBMCs were isolated from the whole blood of three random donors by ficoll gradient centrifugation and plated in a 96 well U-bottom tissue culture plate. Immediately after plating, cells were overlain with either AAV2-empty capsid, AAV2 CMV>null vector, or AAV2>CAG>GFP all pre-treated for one hour with chimeric anti-AAV2 mAb hA20 (murine Fab fused to human Fc), in triplicate. 24 hours later, the supernatant was removed and Luminex performed in duplicate. mIL2RA results are shown. INFα2, CXCL10, MCP3 and INFg results are shown.

FIG. 12: Vector opsonization with chimeric antibodies (human Fc/murine Fab) against AAV9 and AAVrh10 capsids facilitates robust and sensitive measurement of innate immune signaling responses on freshly isolated human PBMCs.

### DETAILED DESCRIPTION

AAV capsid-specific cytotoxic T lymphocytes (CTLs) are formed when CD8⁺ T cells are activated, and are often observed following systemic AAV vector administration in clinical studies. These CTLs can cause loss of transgene expression through destruction of the transduced cells. The AAV capsid-derived peptide targets for these CTLs is derived from an inherent feature of the gene delivery vehicle and cannot be removed. However, there is evidence that signals triggering these CTLs derive from unmethylated CpG motifs in the AAV vector genome. For example, there is marked hypomethylation of CpG dinucleotides present in the genomes of AAV vectors; activation of the toll like receptor 9 (TLR9)-MyD88 innate pathway in pre-clinical studies correlates with the frequency of CpG dinucleotides in AAV vector genomes; and clinical evidence that AAV vectors with less CpGs i.e., lower CpG density trigger lower CTL responses and lead to more durable transgene expression, and reciprocally those with more CpGs i.e., higher CpG density trigger stronger CTL responses and loss of transgene expression. Methods are disclosed herein to determine relevant responses to an AAV composition of interest.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds can also be made by methods known to one of ordinary skill in the art. As used herein, “methods known to one of ordinary skill in the art” may be identified though various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

“Comparable cell” shall mean a cell whose type is identical to that of another cell to which it is compared. Examples of comparable cells can mean cells from the same cell line, or similar biological samples from different patients.

“Inhibiting” a response shall mean either lessening the likelihood of the response onset, or preventing the onset entirely. “Inhibiting” the expression of a gene in a cell shall mean either lessening the degree to which the gene is expressed, or preventing such expression entirely. “Specifically inhibit” the expression of a protein shall mean to inhibit that protein's expression (a) more than the expression of any other protein, or (b) more than the expression of all but 10 or fewer other proteins.

“Treating” a disorder shall mean slowing, stopping or reversing the disorder's progression. In the preferred embodiment, treating a disorder means reversing the disorder's progression, ideally to the point of eliminating the disorder itself. As used herein, ameliorating a disorder and treating a disorder are equivalent.

“Specifically hybridize” to a nucleic acid shall mean, with respect to a first nucleic acid, that the first nucleic acid hybridizes to a second nucleic acid with greater affinity than to any other nucleic acid.

The terms “specific binding,” “specifically binds,” and the like, refer to non-covalent or covalent preferential binding to a molecule relative to other molecules or moieties in a solution or reaction. In some embodiments, the affinity of one molecule for another molecule to which it specifically binds is characterized by a KD (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less). “Affinity” refers to the strength of binding, increased binding affinity being correlated with a lower KD. In an embodiment, affinity is determined by surface plasmon resonance (SPR), e.g. as used by Biacore systems. The affinity of one molecule for another molecule is determined by measuring the binding kinetics of the interaction, e.g. at 25° C.

The terms “subject,” “individual,” and “patient” are used interchangeably herein to refer to a mammal being assessed for treatment and/or being treated. In an embodiment, the mammal is a human, e.g. a human for which gene therapy is desired. Subjects may be human, but also include other mammals, particularly those mammals useful as laboratory models for human disease, e.g. mouse, rat, etc.

The definition of an appropriate patient sample encompasses blood and other liquid samples of biological origin, e.g. PBMC, etc. ; solid tissue samples such as a biopsy specimen or tissue cultures or cells derived there from and the progeny thereof. The definition also includes samples that have been manipulated in any way after their procurement, such as by treatment with reagents; washed; or enrichment for certain cell populations, such as endometrial cells, etc. A sample if interest in bronchial lavage sample. The definition also includes sample that have been enriched for particular types of molecules, e.g., nucleic acids, polypeptides, etc. The term “biological sample” encompasses a clinical sample, and also includes bone marrow, blood, plasma, serum, and the like.

Isolation and cryopreservation of peripheral blood mononuclear cells (PBMC) is common clinical practice. Cryopreservation allows for batching of samples, which is convenient and improves the comparability of data. Cryopreservation also allows cells to be stored for future purposes. PBMC isolation is a common prerequisite to cryopreservation of blood cells; and the most common method for PBMC isolation is density gradient centrifugation using Ficoll-Hypaque, a high molecular weight carbohydrate solution. For example, Cellular Preparation Tubes (CPTs), which contain Ficoll-Hypaque, are used to simplify the standard procedure for density gradient centrifugation in two ways: (1) blood is collected into the same tube that is then used to isolate the PBMC; and (2) the tube is pre-loaded with Ficoll-Hypaque, which is separated by a gel plug so that it is not disturbed by the entry of blood into the tube. After blood draw, the tubes are centrifuged, and the PBMCs and plasma become separated from the erythrocytes and granulocytes by the gel plug. This allows the spun tubes to be shipped, maintaining the PBMC in an isolated environment from the erythrocytes and granulocytes, which may improve their viability and function.

Typical assessment of a PBMC isolation protocol includes monitoring yield and purity. To some degree, the latter can be determined visually, as erythrocyte contamination will redden the pellet of PBMC acquired after centrifugation. This is sometimes seen with CPT, to a greater extent than traditional Ficoll protocols. However, these contaminating erythrocytes are lost after downstream procedures such as cryopreservation, and have not been observed to influence.

Yield is easily determined by either manual or automated cell counting, and has also been reported to be roughly equivalent between CPT and manual Ficoll methods.

“Suitable conditions” shall have a meaning dependent on the context in which this term is used. That is, when used in connection with an antibody, the term shall mean conditions that permit an antibody to bind to its corresponding antigen. When this term is used in connection with nucleic acid hybridization, the term shall mean conditions that permit a nucleic acid of at least 15 nucleotides in length to hybridize to a nucleic acid having a sequence complementary thereto. When used in connection with contacting an agent to a cell, this term shall mean conditions that permit an agent capable of doing so to enter a cell and perform its intended function. In one embodiment, the term “suitable conditions” as used herein means physiological conditions.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

An “effective amount” means the amount of an rAAV antigen or antibody that, when present in an assay, is sufficient to allow for opsonization and uptake of the rAAV particle.

The phrase “determining the treatment efficacy” and variants thereof can include any methods for determining that a treatment is providing a benefit to a subject, or will provide a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typically associated with standard or non-standard treatments of a disease. Determination of treatment efficacy is usually indication and disease specific and can include any methods known or available in the art for determining that a treatment is providing a beneficial effect to a patient. For example, evidence of treatment efficacy can include but is not limited to remission of the disease or indication. Further, treatment efficacy can also include general improvements in the overall health of the subject, such as but not limited to enhancement of patient life quality, increase in predicted subject survival rate, decrease in depression or decrease in rate of recurrence of the indication (increase in remission time). (See, e.g., Physicians' Desk Reference (2010).)

Related vectors. As used herein, related vectors may refer to two or more rAAV vectors that share a high degree of sequence similarity in their genomes. Two related vectors may be at least about 99% identical, at least about 98% identical, at least about 97% identical, at least about 95% identical, at least about 90% identical. Identity may be determined in coding sequences of interest in the rAAV genome, or may be determined over the length of the vector. In some embodiments, two related vectors deliver the same therapeutic gene of interest, but vary in codon usage, or in non-coding sequences.

AAV gene therapy. Utilizing a viral vehicle to deliver genetic material into cells allows direct targeting of pathogenic molecules and restoration of function. The success of adeno-associated virus (AAV)-mediated gene replacement makes therapy with AAV a promising gene therapy strategy. Because AAV is non-pathogenic and cannot reproduce itself without helper viruses, it has served as a primary vehicle for gene therapy. It is a single-stranded DNA virus that stably and efficiently infects a wide variety of cells in multiple tissues.

In some embodiments, a vector is a recombinant adeno-associated virus (AAV) vector. AAV vectors are DNA viruses of relatively small size that can integrate, in a stable and site specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

The application of AAV as a vector for gene therapy has been rapidly developed in recent years. Wild-type AAV can infect, with a comparatively high titer, dividing or non-dividing cells, or tissues of mammal, including human, and also can integrate into in human cells at specific site (on the long arm of chromosome 19) (Kotin et al, Proc. Natl. Acad. Sci. U.S.A., 1990. 87:2211-2215; Samulski et al, EMBO J., 1991. 10:3941-3950 the disclosures of which are hereby incorporated by reference herein in their entireties). AAV vector without the rep and cap genes loses specificity of site-specific integration, but may still mediate long-term stable expression of exogenous genes. AAV vector exists in cells in two forms, wherein one is episomic outside of the chromosome; another is integrated into the chromosome, with the former as the major form. Moreover, AAV has not been found to be associated with any human disease, nor any change of biological characteristics arising from the integration has been observed. There are sixteen serotypes of AAV reported in literature, respectively named AAV1, AAV2 , AAV3, AAV4, AAV5, AAV6, AAV7, AAV8, AAV9, AAV10, AAV11, AAV12, AAV13, AAV14, AAV15, and AAV16, wherein AAV5 is originally isolated from humans (Bantel-Schaal, and H. zur Hausen. Virology, 1984. 134:52-63), while AAV1-4 and AAV6 are all found in the study of adenovirus (Ursula Bantel-Schaal, Hajo Delius and Harald zur Hausen. J. Viral., 1999. 73:939-947).

AAV vectors may be prepared using any convenient methods. Adeno-associated viruses of any serotype are suitable (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. ME Bloom, RMLinden, C RParrish, Eds.) p 5-14, Rudder Arnold, London, UK (2006); and D E Bowles, J E Rabinowitz, R J Samulski “The Genus Dependovirus” (J R Kerr, SF Cotmore. ME Bloom, R M Linden, C R Parrish, Eds.) p 15-23, Rudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and W0/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCTIUS2005/027091, the disclosure of which is herein incorporated by reference in its entirety. The use of vectors derived from the AAVs for transferring genes in vitro and in vivo has been described (See e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in the methods of the invention are encapsidated into a virus particle (e.g. AAV virus particle including, but not limited to, AAV1, AAV2 , AAV3, AAV4, AAV5, AAV6, AAV7, AAV8, AAV9, AAVIO, AAVII, AAV12, AAV13, AAV14, AAV15, and AAV16). Accordingly, the invention includes methods for assay of a recombinant virus particle (recombinant because it contains a recombinant polynucleotide) comprising any of the vectors described herein. Methods of producing such particles are known in the art and are described in U.S. Pat. No. 6,596,535.

AAV test samples may include crude preparations, e.g. a cell lysate, a genome sample, or may comprise purified or partially purified capsids(s) or particles. The term substantially pure means a preparation which contains at most 5% by weight of other material with which it is natively associated. In some embodiments the genomic sequences of the AAV sample(s) differ in the genomic sequence of the virus, and/or the sequence of polypeptides in the capsid. Such sequences include analogs and variants produced by recombinant methods wherein such nucleic acids and polypeptide sequences are modified by substitution, insertion, addition, and/or deletion of one or more nucleotides in the nucleic acid sequence to cause the substitution, insertion, addition, and/or deletion of one or more amino acid residues in the recombinant polypeptide.

Pattern Recognition Receptors. Pattern recognition receptors, or PRRs, are proteins expressed by cells of the innate immune system to identify pathogen-associated molecular patterns, or PAMPs, which are associated with microbial pathogens or cellular stress. They may also be referred to as pathogen recognition receptors or primitive pattern recognition receptors. The molecules that are recognized by a given PRR are called PAMPs: pathogen-associated molecular patterns and include bacterial carbohydrates (e.g. lipopolysaccharide or LPS, mannose), nucleic acids (e.g. bacterial or viral DNA or RNA), bacterial peptides (flagellin, ax21), peptidoglycans and lipotechoic acids (from Gram positive bacteria), N-formylmethionine, lipoproteins and fungal glucans.

PRRs are classified according to their ligand specificity, function, localization and/or evolutionary relationships. On the basis of function, PRRs may be divided into endocytic PRRs or signaling PRRs. Signaling PRRs include the large families of membrane-bound Toll-like receptors and cytoplasmic NOD-like receptors, retinoid acid-inducible gene I-like receptors, and DNA sensors such as such AIM2. Endocytic PRRs promote the attachment, engulfment and destruction of microorganisms by phagocytes, without relaying an intracellular signal. These PRRs recognize carbohydrates and include mannose receptors of macrophages, glucan receptors present on all phagocytes and scavenger receptors that recognize charged ligands, are found on all phagocytes and mediate removal of apoptotic cells.

Recognition of extracellular or endosomal pathogen-associated molecular patterns is mediated by an array of transmembrane proteins known as toll-like receptors (TLRs). TLRs are members of the IL-1 receptor superfamily. Toll-like receptors trigger a series of mechanisms leading to the synthesis and secretion of cytokines and activation of other host defense programs that are crucial to the development of innate or adaptive immune responses. In mammals, these receptors have been assigned numbers 1 to 11 (TLR1-TLR11). Interaction of TLRs with their specific PAMP induces NF-KB signaling and MAP kinase pathway and therefore the secretion of pro-inflammatory cytokines and co-stimulatory molecules. Molecules released following TLR activation signal to other cells of the immune system making TLRs key elements of innate immunity and adaptive immunity.

Toll-like receptor 9 responses are of particular interest for the assay methods of the disclosure. TLR9 is expressed in immune system cells including dendritic cells, macrophages, natural killer cells, and other antigen presenting cells. It binds DNA (preferentially DNA containing unmethylated CpGs of bacterial or viral origin), and triggers signaling cascades that lead to a pro-inflammatory cytokine response. TLR9 is usually activated by unmethylated CpG sequences in DNA molecules. Once activated, TLR9 moves from the endoplasmic reticulum to the Golgi apparatus and lysosomes, where it interacts with MyD88, the primary protein in its signaling pathway. CpG sites are relatively rare in vertebrate genomes in comparison to bacterial genomes or viral DNA.

TLR9 signals lead to activation of the cells initiating pro-inflammatory reactions that result in the production of cytokines, including for example type-I interferons, IL-6, TNF and IL-12. In humans, the type I IFN system consists of a family of IFN proteins encoded by at least 13 IFN alpha (IFNA) subtype genes (IFN-a1, -a2, -a4, -a5, -a6, -a7, -a8, -a10, -a13, - a14, -a16, -a17 and -a21), and one IFN beta gene (IFNB), one IFN-Epsilon gene, one IFN-Kappa gene, and IFN-Omega gene, all of which bind to the type I interferon receptor composed of the IFNAR1 and IFNAR2 chains.

Antibody opsonization. In nature phagocytic immune cells internalize pathogens, often initiating innate immune responses and signaling pathways that build to adaptive responses. Once adaptive humoral responses are established, antibodies can specifically bind to pathogens, thereby a) neutralizing pathogen interactions with host cells, b) increasing phagocytotic uptake through agglutination and opsonization, and c) initiating complement fixation and membrane attack complex formation, leading to cell lysis and immune recruitment.

The phenomenon of antibody opsonization to increase the uptake of rAAV by phagocytotic cells can reveal fundamental immune responses to rAAV in PBMCs. Incubation of an AAV test sample with opsonizing antibodies, e.g. human, humanized, or chimeric monoclonal antibodies specific for AAV and comprising an Fc region that binds to human Fc receptors, e.g. human FcγRs, increase uptake of the AAV by plasmacytoid dendritic cells (pDCs) in a PBMC composition, and can thereby increase the immune response to the virus. The immune response may include, without limitation, type 1 interferon secretion in response to TLR9 stimulation by rAAV comprising genomic CpG-dense conditions, compared to genomic CpG-free controls.

The Fcγ receptors (FcγR) are the most important Fc receptors for inducing phagocytosis of opsonized particles. This Fc family includes several members, FcγRI (CD64), FcγRIIA (CD32), FcγRIIB (CD32), FcγRIIIA (CD16a), FcγRIIIB (CD16b), which differ in their antibody affinities due to their different molecular structure.

Antibodies, also referred to as immunoglobulins, conventionally comprise at least one heavy chain and one light, where the amino terminal domain of the heavy and light chains is variable in sequence, hence is commonly referred to as a variable region domain, or a variable heavy (VH) or variable light (VH) domain. The two domains conventionally associate to form a specific binding region, although as well be discussed here, a variety of non-natural configurations of antibodies are known and used in the art. Antibodies may comprise an Fc region sequence.

A “functional” or “biologically active” antibody or antigen-binding molecule is one capable of exerting one or more of its natural activities in structural, regulatory, biochemical or biophysical events. For example, a functional antibody or other binding molecule may have the ability to specifically bind an antigen and the binding may in turn elicit or alter a cellular or molecular event such as signaling transduction or enzymatic activity. A functional antibody or other binding molecule may also block ligand activation of a receptor or act as an agonist or antagonist. The capability of an antibody or other binding molecule to exert one or more of its natural activities depends on several factors, including proper folding and assembly of the polypeptide chains.

The term “antibody” herein is used in the broadest sense and specifically covers monoclonal antibodies, polyclonal antibodies, monomers, dimers, multimers, multispecific antibodies (e.g., bispecific antibodies), heavy chain only antibodies, three chain antibodies, single chain Fv, nanobodies, etc., and also include antibody fragments, so long as they exhibit the desired biological activity (Miller et al (2003) Jour. of Immunology 170:4854-4861). Antibodies may be murine, human, humanized, chimeric, or derived from other species.

The term antibody may reference a full-length heavy chain, a full length light chain, an intact immunoglobulin molecule; or an immunologically active portion of any of these polypeptides, i.e., a polypeptide that comprises an antigen binding site that immunospecifically binds an antigen of a target of interest or part thereof, such targets including but not limited to, cancer cell or cells that produce autoimmune antibodies associated with an autoimmune disease. The immunoglobulin disclosed herein can comprise an Fc sequence of any type (e.g., IgG, IgE, IgM, IgD, and IgA), class (e.g., IgG1, IgG2, IgG3, IgG4, IgA1 and IgA2) or subclass of immunoglobulin molecule, including engineered subclasses with altered Fc portions that provide for reduced or enhanced effector cell activity. The immunoglobulins can be derived from any species. In one aspect, the immunoglobulin is of largely human origin.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al (1991) Sequences of Proteins of Immunological Interest, 5th Ed. Public Health Service, National Institutes of Health, Bethesda, Md.). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region may comprise amino acid residues from a “complementarity determining region” or “CDR”, and/or those residues from a “hypervariable loop”. “Framework Region” or “FR” residues are those variable domain residues other than the hypervariable region residues as herein defined.

Variable regions of interest include 3 CDR sequences, which may be obtained from available antibodies with the desired specificity, or may be obtained from antibodies developed for this purpose. One of skill in the art will understand that a number of definitions of the CDRs are commonly in use, including the Kabat definition (see “Zhao et al. A germline knowledge based computational approach for determining antibody complementarity determining regions.” Mol Immunol. 2010;47:694-700), which is based on sequence variability and is the most commonly used. The Chothia definition is based on the location of the structural loop regions (Chothia et al. “Conformations of immunoglobulin hypervariable regions.” Nature. 1989;342:877-883). Alternative CDR definitions of interest include, without limitation, those disclosed by Honegger, “Yet another numbering scheme for immunoglobulin variable domains: an automatic modeling and analysis tool.” J Mol Biol. 2001;309:657-670; Ofran et al. “Automated identification of complementarity determining regions (CDRs) reveals peculiar characteristics of CDRs and B cell epitopes.” J Immunol. 2008;181:6230-6235; Almagro “Identification of differences in the specificity-determining residues of antibodies that recognize antigens of different size: implications for the rational design of antibody repertoires.” J Mol Recognit. 2004;17:132-143; and Padlanet al. “Identification of specificity-determining residues in antibodies.” Faseb J. 1995;9:133-139., each of which is herein specifically incorporated by reference.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method.

The antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies of interest herein include “primatized” antibodies comprising variable domain antigen-binding sequences derived from a non-human primate (e.g., Old World Monkey, Ape etc) and human constant region sequences.

An “intact antibody chain” as used herein is one comprising a full length variable region and a full length constant region. An intact “conventional” antibody comprises an intact light chain and an intact heavy chain, as well as a light chain constant domain (CL) and heavy chain constant domains, CH1, hinge, CH2 and CH3 for secreted IgG. Other isotypes, such as IgM or IgA may have different CH domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis (ADCP); and down regulation of cell surface receptors. Constant region variants include those that alter the effector profile, binding to Fc receptors, and the like.

Depending on the amino acid sequence of the constant domain of their heavy chains, intact antibodies can be assigned to different “classes.” There are five major classes of intact immunoglobulin antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two clearly distinct types, called K and A, based on the amino acid sequences of their constant domains. Opsonizing antibodies of interest for use in the methods of the disclosure opsonize AAV, e.g. rAAV, and bind to Fc receptors, e.g. FcγR. Typically the opsonizing antibodies will bind to human FcR, for use with human PBMC assays. However in some circumstances it ca be useful to perform assays with non-human PBMC, where the opsonizing antibodies are selected to comprise an Fc region that will bind to the FcR present on the non-human PBMC.

In some embodiments, human antibodies are used, which may include monoclonal and polyclonal antibodies, for example AAV capsid seropositive blood, plasma or sera taken from human subjects, which subjects may have been exposed to rAAV. Alternatively, where the assay PBMC are non-human, AAV capsid seropositive blood, plasma or taken from research animals subsequent to rAAV administration can be used as opsonizing antibodies.

In some embodiments, AAV capsid specific monoclonal antibodies, or a cocktail of AAV capsid specific monoclonal antibodies are used as opsonizing antibodies. The monoclonal antibody will comprise an Fc region suitable for the PBMC in the assay, e.g. a human, humanized or chimeric antibody may be chosen or made in order to bind to FcR present on human PBMC.

AAV specific antibodies are known in the art and commercially available, for example and without limitation, Progen AAV capsid protein antibodies specific for VP1, VP1/VP2 and VP1/VP2/VP3. AAV particle antibodies are also available, which recognize conformational epitopes of the viral capsid proteins that are only present on fully assembled capsids. These epitopes consist of distant amino acids either of the same protein or of different proteins that are in close proximity, and they might vary across the different serotypes. Specificities include, for example, AAV1; AAV2 ; AAV3; AAV4; AAV5; AAV6; AAV7; AAV8; AAV9; AAVrh10; AAV12; AAVrh74; Anc80. Engineered human anti-AAV antibodies are disclosed in, for example US20180179265A1, herein specifically incorporated by reference. Alternatively, mouse anti-AAV antibodies, e.g. A20, can be modified by chimerization or humanization for use with human PBMC.

### Assay Methods

The methods of the invention provide improved assays for detecting the presence of an AAV-specific cell-mediated immune response, which response is predictive of the immune response produced following introduction of the AAV to an individual. In the methods of the invention, a sample comprising immune cells, e.g. dendritic cells, monocytes, T cells, B cells, NK cells, etc., usually a blood sample or derivative thereof, including without limitation a peripheral blood mononuclear cell sample, is contacted with an effective amount of a test AAV composition in the presence of an effective amount of AAV-specific opsonizing antibodies. The opsonizing antibodies specifically bind to AAV and comprise an Fc region sequence that binds to Fc receptors present on the immune system cells. The sample is incubated for a period of time sufficient to allow for uptake of the opsonized AAV, activation of responsive T cells; and release of IFN-α or other cytokine or immune effector molecule(s). The level of IFN-α or other cytokine or immune effector molecule(s) produced by the immune cells is indicative of the level of cell mediated responsiveness to the AAV composition. The opsonizing antibody is shown herein to increase the sensitivity of the assay.

A sample of immune cells may be obtained from an individual of interest, e.g. a candidate for gene therapy. Alternatively a sample may be obtained from one or a panel of individuals, or may be commercially obtained, for use in general assessment of responsiveness to the test AAV. Samples, as used herein, typically refer to a cellular sample comprising at least T cells and antigen-presenting cells, e.g. dendritic cells, monocytes, B cells, etc. Blood samples are conveniently used as a source of complex cell populations, although for certain purposes other cellular samples may find use, bone marrow, synovial fluid, cerebrospinal fluid, bronchial washes, tissue samples, and the like. The term blood samples may also refer to a mononuclear cell fraction, e.g. PBMC, which may be isolated by conventional methods. The sample may be used directly, or diluted as appropriate. Assays may be performed in any suitable physiological buffer, e.g. PBS, normal saline, HBSS, dPBS, etc., and may further contain a sugar, e.g. dextrose, heparin, etc., as known in the art.

PBMC immune cells include T lymphocytes, B lymphocytes, natural killer cells, monocytes, and dendritic cells, and can be obtained through minimally invasive blood draws from research animals, healthy human donors, and prospective rAAV GT recipients. Once isolated though gradient centrifugation, PBMCs can be frozen or kept alive in cell culture dishes where, after exposure to stimuli, immune responses can be measured by various assays.

The sample is usually deposited into a blood collection tube. A blood collection tube includes a blood draw tube or other similar vessel. Conveniently, when the sample is whole blood, the blood collection tube is heparinized. Alternatively, heparin is added to the tube after the blood is collected. Notwithstanding that whole blood is the preferred and most convenient sample, the present invention extends to other samples containing immune cells such as lymph fluid, cerebral fluid, tissue fluid and respiratory fluid including nasal and pulmonary fluid.

Cells in the cellular sample may be present at a concentration of from about 10⁴, 2.5×10⁴, 5×10⁴, 7.5×10⁴, 10⁵, 2.4×10⁵, 5×10⁵, 7.5×10⁵, 10⁶ mononuclear cells/ml. Each assay well may comprise from about1 μl to about 1 ml of cells, e.g. 1 μl, 10 μl, 100 μl, 500 μl, 1 ml or more. The MOI of rAAV to cells may be from about 10² to about 10⁶, e.g. 10², 10³, 10⁴, 10⁵ , 10⁶, or more.

The cellular sample is incubated with a target AAV composition or compositions. One or a panel of rAAV can be tested. Suitable positive and negative controls are usually included, e.g. as a negative control empty AAV capsids have found use. For example, the number of AAV in an assay well may range from about 10⁶, 10⁷, 10⁸, 10⁹, 10¹⁰, 10¹¹, or more.

The AAV test composition may be pre-incubated with an effective amount of opsonizing antibody, or the antibody may be added essentially simultaneously into the assay with the test AAV composition. The antibody concentrations will vary with the specific antibosy being used, but will generally be sufficient to provide an enhancing effect. For example, a suitable monoclonal or polyclonal antibody may be provided at a concentration of from about 0.1 μg/ml, about 0.5 μg/ml, about 1 μg/ml, about 5 μg/ml, about 50 μg/ml, about 100 μg/ml, about 500 μg/ml, or more.

The incubation step may be from 5 to 50 hours, more preferably 5 to 40 hours and even more preferably 8 to 24 hours or a time period in between.

Following incubation of the cellular sample with antigen and opsonizing antibody, the release of immune effector molecules, e.g. IFN-α, etc., may be measured by any convenient method. Methods of interest include ELISA or other assay for quantitating the concentration of release effector molecule in the sample buffer. Alternatively the level of mRNA encoding the immune effector molecule can be measured.

For example, a first antibody having specificity for the immune effector molecule of interest is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, spheres, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-120 minutes or where more convenient, overnight) and under suitable conditions (e.g. for about 20° C. to about 40° C.) to allow binding of any molecule present in the antibody sample. Following the incubation period, the antibody solid phase is washed and dried and incubated with a second antibody specific for a portion of the antigen, or for the first antibody. The second antibody is usually linked to a reporter molecule which is used to indicate binding.

Both polyclonal and monoclonal antibodies are obtainable by immunization with the immune effectors or antigenic fragments thereof and either type is utilizable for immunoassays. The methods of obtaining both types of sera are well known in the art. Polyclonal sera are less preferred but are relatively easily prepared by injection of a suitable laboratory animal with an effective amount of the immune effector, or antigenic part thereof, collecting serum from the animal and isolating specific sera by any of the known immunoadsorbent techniques. The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art.

In some cases, a competitive assay will be used. In addition to the patient sample, a competitor to the antibody is added to the reaction mix. The competitor and the antibody compete for binding to the polypeptide. Usually, the competitor molecule will be labeled and detected as previously described, where the amount of competitor binding will be proportional to the amount of Immune present. The concentration of competitor molecule will be from about 10 times the maximum anticipated Immune concentration to about equal concentration in order to make the most sensitive and linear range of detection.

A reporter molecule provides an analytically identifiable signal that allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules. In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable colour change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-antigen complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.

There are many variations to these assays. One particularly useful variation is a simultaneous assay where all or many of the components are admixed substantially simultaneously.

An alternative method utilizes a filter immunoplaque assay, otherwise called the enzyme-linked immunospot assay (ELISPOT). These assays take advantage of the relatively high concentration of a given cell product (such as a cytokine) in the environment immediately surrounding the protein-secreting cell. These cell products are captured and detected using high-affinity antibodies. The ELISPOT assay is reviewed in “Current Protocols in Immunology, Unit 6.19, pages 6.19. 1-8”.

The ELISPOT assay involves coating a purified cytokine-specific antibody to a membrane-backed microtiter plate; blocking the plate to prevent non-specific absorption of any other proteins; incubating the cytokine-secreting cells with appropriate reagents; removal of cells and reagents; adding a labeled second anti-cytokine antibody; and detecting the antibody-cytokine complex on the membrane. The ELISPOT assay thus utilizes two high-affinity cytokine-specific antibodies directed against different epitopes on the same cytokine molecule: either two monoclonal antibodies or a combination of one monoclonal antibody and one polyvalent antiserum. ELISPOT generates spots based on a colorimetric reaction that detects the cytokine secreted by a single cell. The spot represents a “footprint” of the original cytokine-producing cell. Spots are permanent and can be quantified visually, microscopically or electronically. Detection methods using fluorescence labels are also practiced in the art. The ELISPOT assay can be used in a clinical setting, where for example, each kit is able to assay 24 patient samples at one time (using 4 wells per sample in a 96 well plate).

An alternative method may utilize a polynucleotide based assay, e.g. PCR, etc., tod determine the level of mRNA encoding the effector polypeptide, e.f. IFNα, produced by the immune cells in the assay.

The present invention further contemplates a kit for assessing the immune responsivenss to an AAV composition. The kit is conveniently in compartmental form with one or more compartments adapted to receive a sample from a subject such as whole blood. That compartment or another compartment may also be adapted to contain heparin where the sample is whole blood with or without a simple sugar such as dextrose. The simple sugar may also be maintained in a separate container. The kit will generally include an effective dose of an opsonizing antibody, and may frequently include positive and negative control samples.

Generally, the kit is in a form which is packaged for sale with a set of instructions. The instructions would generally be in the form of a method for measuring a response in a subject, said method comprising collecting a sample from said subject wherein said sample comprises cells of the immune system which are capable of producing immune effector molecules following stimulation by AAV, incubating said sample with an antigen and then measuring the presence or elevation in level of an immune effector molecule wherein the presence or level of said immune effector molecule is indicative of the capacity of said subject to mount a cell-mediated immune response.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. It is also understood that the terminology used herein is for the purposes of describing particular embodiments

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the appended claims.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLE 1

Toll like receptor 9 (TLR9) innate immune receptor-mediated responses to AAV gene therapy are emerging as a critical concern. Unmethylated CpG dinucleotides, abundant in unmodified AAV vector genomes, are TLR9 ligands, and are inversely correlated with clinical success. TLR9 signaling leads to type 1 interferon secretion and subsequent Cytotoxic T Lymphocyte (CTL) activation and may contribute to severe adverse events including complement activation associated thrombotic microangiopathy. Unfortunately, many current methods to measure TLR9 responses rely upon limited rodent or cell line models, cannot discriminate between related vectors, and lack an effector cell readout. Here, we present a human PBMC-based assay capable of differentiating between closely related AAV vectors based on the magnitude of the type 1 interferon responses they elicit, and subsequent T cell activation.

HEK reporter cells are widely used to predict TLR9 responses to candidate AAV gene therapy vectors. While this assay can sufficiently measure TLR9 activation by oligonucleotides, it is poorly stimulated by tested AAV vector doses up to 10⁴ MOI. The secretion of type 1 interferons by murine PBMCs is minimal, and vectors are indistinguishable from each other in preliminary experiments. Furthermore, AAV vectors can elicit statistically significant immune signals in human PBMCs, but this is highly donor dependent, and differences are difficult to measure between related vectors.

We hypothesized that post AAV gene therapy serum will opsonize candidate vectors, increasing Fc receptor (FcR)-mediated uptake and type 1 IFN signaling. We obtained serum from patients before and after AAV9 gene therapy and observed dose-dependent increases in post therapy serum-mediated neutralization of AAV2 transduction in cell culture, compared to pre-gene therapy serum. We then exposed PBMCs from three healthy donors to empty AAV capsids or AAV2-CAG>GFP. We observed a 19.1 pg increase in INF1α secretion after 24 hours in PBMCs exposed to the GFP vector, compared to empty capsid. However, when we first incubated vectors with post gene therapy serum, we observed a >3000% increase in signal, with 727.27 pg secreted. Opsonization did not significantly influence empty capsid signal.

We then interrogated the serum-mediated effect for FcR-dependence and found that when PBMCs were pretreated with FcR blocking antibodies, the post gene therapy serum-mediated signal increase was blunted. Interestingly, flow cytometry analysis of human PBMCs exposed to post-gene therapy serum-incubated vectors after 72 hrs showed increased expression of T-cell activation markers consistent with CTL activation.

As post AAV gene therapy seropositive human serum is predicted to be variable, we next attempted opsonization of AAV2 empty capsids, AAV2-CMV>null, and AAV2-CAG>GFP with AAV monoclonal antibodies. We were pleasantly surprised to observe opsonized vector-induced INF1α responses from PBMC donors that were otherwise unresponsive to stimulation, even by oligonucleotide positive control. Moreover, INF1α responses, otherwise indistinguishable by ELISA, correspondingly increased with exposure to unmethylated genomic CpGs (p<0.0001) when vectors were first opsonized, and this effect was recapitulated in Luminex readouts for INFγ, IL1Rα, IL6, IL8, CXCL10, MCP2 and MCP3. Opsonization of AAV vectors prior to exposure on human PBMCs enables, for the first, a human-based assay of TLR9-downstream cytokines that can distinguish between vectors that vary by genomic CpG content; and provides an important tool for developing vectors that evade TLR9 recognition.

In nature phagocytic immune cells internalize pathogens, often initiating innate immune responses and signaling pathways that build to adaptive responses. Once adaptive humoral responses are established, antibodies can specifically bind to pathogens a) neutralizing pathogen interactions with host cells, b) increasing phagocytotic uptake through agglutination and opsonization, and c) initiating complement fixation and membrane attack complex formation, leading to cell lysis and immune recruitment. The phenomenon of antibody opsonization can be used to increase the uptake of rAAV by phagocytotic cells, which can reveal fundamental immune responses to rAAV in PBMCs.

Once isolated though gradient centrifugation, PBMCs can be kept alive in cell culture dishes where, after exposure to stimuli, immune responses can be measured by various assays including western blot, flow cytometry, qPCR, ELISA, Luminex, and others. However, signal strength has generally been low and artifact high in response to rAAV, and current methods have not been capable of reliably distinguishing the immune responses elicited by subtly differing vectors, such as those that vary only in genomic sequence.

We hypothesized that rAAV incubation with post-gene therapy (GT) serum would increase uptake by plasmacytoid dendritic cells (pDCs) in PBMCs, and thereby increase type 1 interferon secretion in rAAV genomic CpG-dense conditions, compared to genomic CpG-free controls. Unmethylated CpG dinucleotides are TLR9 ligands abundant in unmodified rAAV vector genomes, and are inversely correlated with clinical success. TLR9 signaling leads to type 1 interferon secretion and subsequent CTL activation and may contribute to severe adverse events including complement activation associated thrombotic microangiopathy.

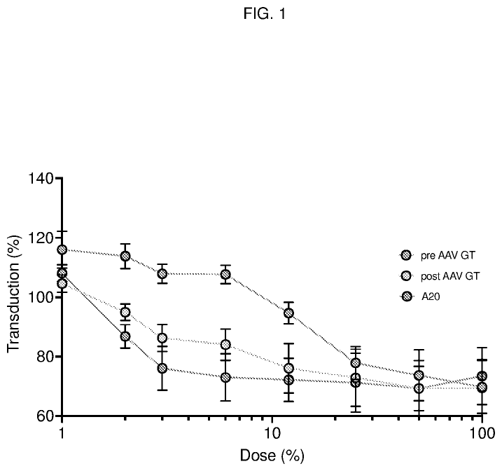

We obtained serum from patients before and after rAAV9 GT and confirmed seroconversion when we observed dose-dependent increases in post-GT serum mediated neutralization of rAAV2 transduction in cell culture, compared to pre-GT serum (FIG. 1). These data additionally suggest antibody cross reactivity between rAAV2 and rAAV9 serotypes.

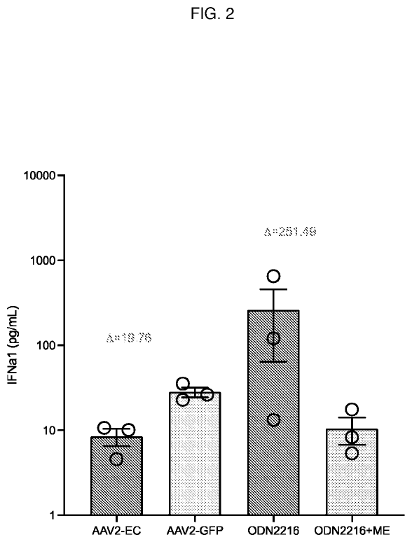

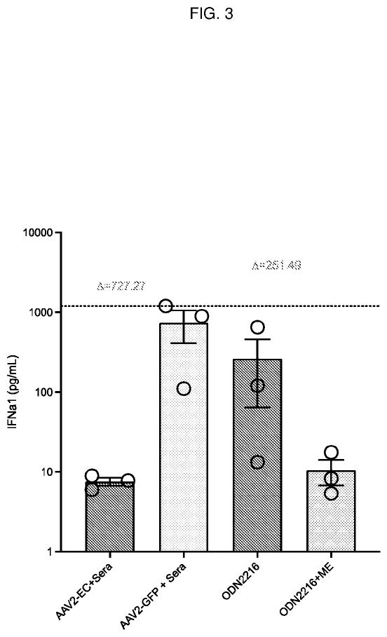

We then exposed PBMCs from three healthy donors to either rAAV2 carrying a CAG promoter driving GFP, which is a genome rich in unmethylated CpG pathogen associated molecular pattern (PAMP) ligands predicted to stimulate TLR9-mediated innate immune responses, or rAAV2-empty capsid counterparts without significant genomes. In controls we observed a 19.1 pg increase in INFα1 secretion after 24 hrs in PBMCs exposed to the GFP vector, compared to empty capsid (FIG. 2). However, when we first incubated rAAVs with post-GT serum, we observed a ~3000% increase in signal, with 727.27 pg secreted INFα1(FIG. 3 ). Consistent with expectations, opsonization did not significantly influence signal in empty capsid conditions as INFα1 secretion at 24hrs is thought to be dependent on genomic PAMPs rather than protein PAMPs.

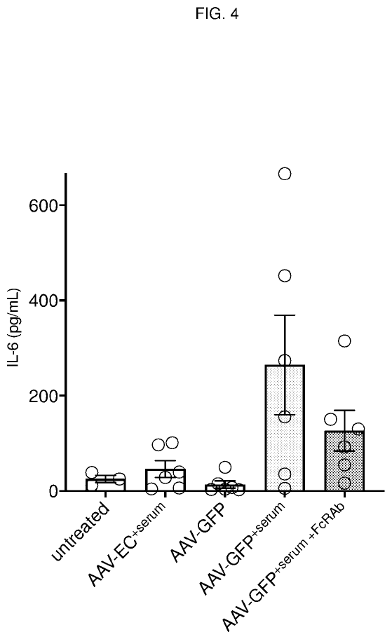

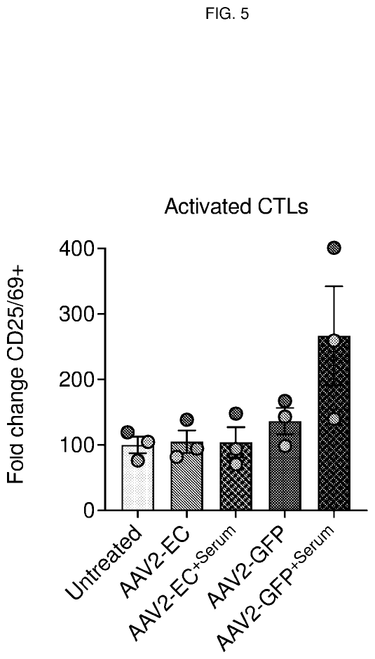

We next interrogated the serum-mediated effect for dependence on interactions between serum antibodies and cellular Fc receptors (FcR), which was confirmed when PBMC pretreatment with FcR blocking antibodies blunted post-GT serum-mediated signal increases (FIG. 4). Moreover, flow cytometry analysis revealed that compared with exposure to rAAVs without serum treatment, human PBMCs exposed to post-GT serum-incubated rAAVs showed increased expression of T cell activation markers after 72 hrs (FIG. 5), consistent with CTL effector cell activation downstream of INFα1 secretion by pDCs.

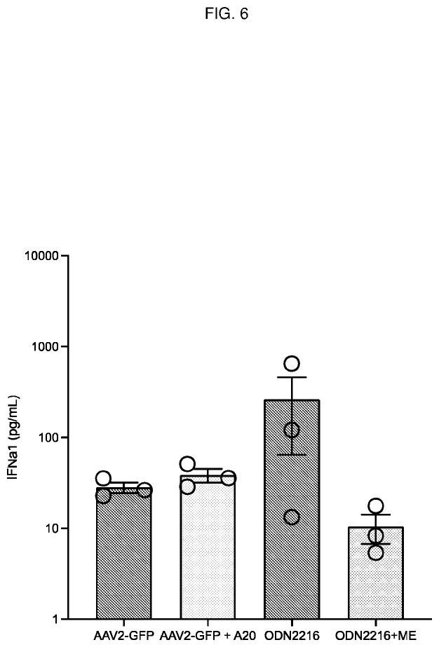

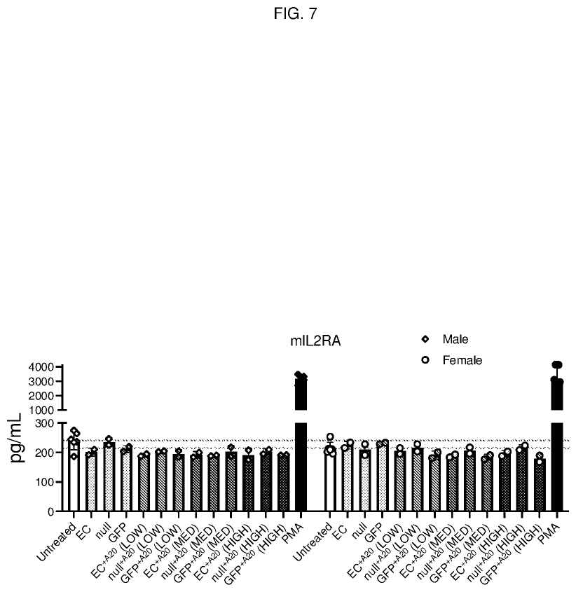

As post-GT human serum is predicted to be variable, we next investigated opsonization of rAAV2-empty capsids, and rAAV2-CAG; GFP with commercially available anti-rAAV monoclonal antibodies. The murine monoclonal A20 antibody is well established in rAAV research and known to bind and neutralize rAAV transduction. However, A20-preincubation-mediated increases in INFα1 secretion were statistically insignificant compared to non-incubated rAAV controls (FIG. 6). Furthermore, preliminary experiments exposing murine PBMCs to rAAVs incubated with increasing A20 concentrations suggest opsonization does not enhance signal to noise ratio in this context (FIG. 7). We speculate rAAV uptake by human pDCs could not be facilitated by A20 due to the incompatibility of the murine IgG3 Fc region with the human FcR, as relevant mouse and human proteins share only 63% identity determined by NCBI BLAST. We further speculate that the inability of A20 to significantly enhance murine PBMC signal to noise ratios is due to inherent limitations of the murine model.

We therefore hypothesized that engineering an IgG antibody combining a human IgG1 Fc subunit with the murine A20 IgG3 Fab subunits, which contain the complementarity determining regions, would achieve a chimeric monoclonal antibody (hA20) capable of consistently and reliably enhancing signal to noise ratios after stimulation of human PBMCs with hA20-incubated rAAV.

We investigated by stimulating human PBMCs from three healthy donors with three rAAVs: rAAV2-empty capsids, rAAV2-CMV null vectors carrying intermediate concentrations of genomic CpGs, and rAAV2-CAG GFP, either with or without chimeric monoclonal hA20 incubation prior to stimulation. For each donor, 2.5×10⁴ PBMCs suspended in 100 ul media (RPMI+10%FBS+1%PennStrep) were plated in a U bottom cell culture treated 96 well plate. Immediately, 100 ul of media containing 2.5×10⁹ rAAVs (MOI=10⁴) with or without 1 hr preincubation with 12.75 mg hA20 were added to wells in triplicate. TLR9 agonist ODN2216 and CpG-free ODN2216 were added to control wells. 24 hrs later, the plate was centrifuged at 400 rfu for 7 mins to pellet the cells and supernatant was collected and assayed for INFα1 secretion.

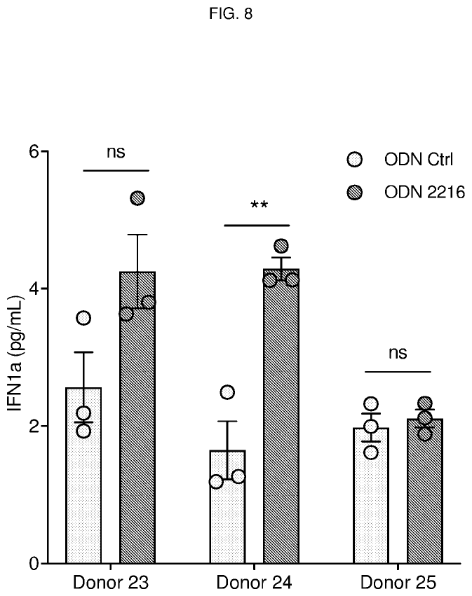

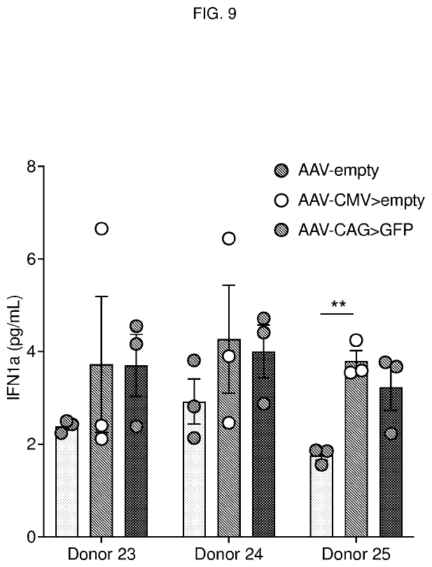

As expected, immune responses were highly donor dependent; while supernatant from all three donors contained increased INFα1 in ODN2216 positive control conditions compared to CpG-free ODN2216, statistical significance was limited to one donor (FIG. 8). Furthermore, the differences in PBMC supernatant INFα1concentrations, elicited by the three untreated rAAVs, was statistically insignificant in 8/9 comparisons (FIG. 9). However, we observed hA20 opsonized vector induced INFα1 responses from PBMC donors that were previously unresponsive to stimulation by either untreated rAAVs or oligonucleotide positive controls.

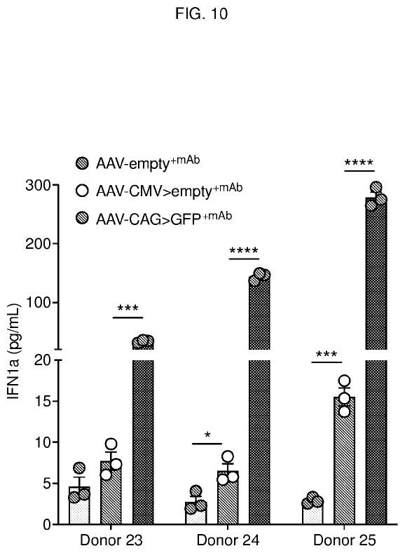

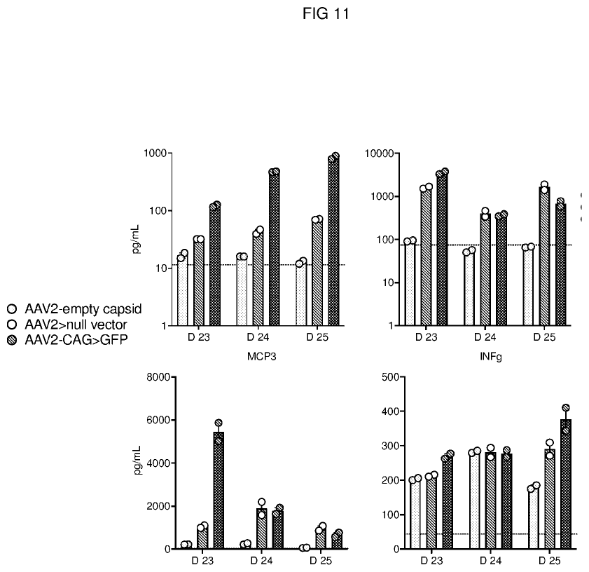

Moreover, INFα1 responses correspondingly increased with exposure to unmethylated genomic CpGs (p<0.0001) when vectors were first hA20-incubated (FIG. 10); similar effects were observed in Luminex readouts for MCP3, CXCL10, and INFγ (FIG. 11).

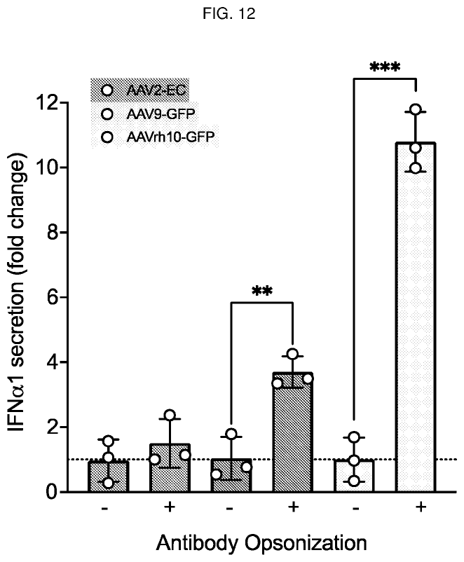

Shown in FIG. 12, peripheral blood mononuclear cells (PBMCs) were isolated from random healthy donors using Ficoll density gradient centrifugation of whole blood. PBMCs suspended in complete media (RPMI+FBS+P/S) were plated in U-bottom tissue culture treated 96 well plates at 250,000 cells/well, in triplicate for each experimental condition. In parallel, AAV vectors were preincubated with capsid specific chimeric antibodies (human Fc/murine Fab) and then added to PBMCs for a final concentration of 2.5×10⁹ viral genomes (MOI 10⁴) and 2 μg of antibodies per well. Seeded plates were incubated under standard human cell culture conditions for 24 hours, after which cells were centrifuged at 600 g for 5 minutes and supernatant collected for ELISA analysis of IFN secretion. These data show that vector opsonization with chimeric antibodies (human Fc/murine Fab) against AAV9 and AAVrh10 capsids facilitates robust and sensitive measurement of innate immune signaling responses on freshly isolated human PBMCs.

Our method can be employed to elucidate human immune responses to rAAV exhaustively, through TLR9, or though other receptors such as TLR2 and TLR4 where viral protein is a ligand. Opsonization of rAAV vectors prior to exposure on human PBMCs enables, for the first time, a human-based assay that can distinguish closely related vectors by innate immune responses, revealing an important tool for developing vectors that evade immune recognition, verifying production lot consistency, and modeling immune responses to rAAV using primary cells taken from prospective GT recipients.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method for determining the immunostimulatory potential of an AAV composition, the method comprising:
combining in an assay (i) a test AAV composition; (ii) opsonizing anti-AAV antibodies; and (iii) a population of immune cells;
incubating for a period of time sufficient to activate immune cells to release immune effector molecules;
measuring the level of released immune effector molecules;
wherein the level of immune effector molecule produced by the immune cells is indicative of the level of the immunostimulatory potential of the test AAV composition.

2. The method of claim 1, wherein the AAV composition comprises recombinant AAV capsids.

3. The method of claim 1, wherein a plurality of assays is performed.

4. The method of claim 3, wherein a plurality of test AAV compositions are assayed, optionally where the plurality of test AAV compositions differ in the virus genomic sequence.

5. (canceled)

6. The method of claim 1, wherein the population of immune cells are peripheral blood mononuclear cells (PBMC), optionally human PBMC

7. (canceled)

8. The method of claim 6, wherein the opsonizing anti-AAV antibodies comprise human Fc region sequences.

9. The method of claim 6, wherein the PBMC are obtained from an individual, to determine the AAV immunostimulatory potential prior to therapeutic administration of the AAV.

10. The method of claim 6, wherein the AAV composition is a candidate for therapeutic gene therapy or a vaccine candidate.

11. (canceled)

12. The method of claim 6, wherein the PBMC are non-human mammalian cells.

13. The method of claim 1, wherein the immune effector molecule is a cytokine, optionally a pro-inflammatory cytokine, optionally a Type I interferon, optionally interferon alpha (IFN-α).

14-16. (canceled)

17. The method of claim 1, wherein the opsonizing antibody comprises AAV capsid-seropositive blood, plasma or sera taken from human subjects, subsequent to rAAV administration.

18. The method of claim 1, wherein the the AAV-specific opsonizing antibody comprises AAV capsid-seropositive blood, plasma or taken from research animals subsequent to rAAV administration.

19. The method of claim 1, wherein the AAV-specific opsonizing antibody comprises AAV capsid-specific monoclonal antibodies.

20. The method of claim 1, wherein the AAV-specific opsonizing antibody comprises AAV capsid-specific monoclonal antibodies engineered or selected for binding through their Fc domain to human Fc receptors, or for binding to non-human Fc receptors.

21. The method of claim 20, wherein the monoclonal antibodies are chimeric comprising a human Fc region sequence, humanized, or human.

22. The method of claim 1, wherein or level of immune effector protein is determined by the presence of the protein or by presence of an mRNA encoding the protein.

23. The method of claim 22, wherein measuring the level of released immune effector molecules is performed by western blot, flow cytometry, PCR, ELISA, Luminex, ELISpot, cyTOF, or tetramer assay.

24. The method of claim 1, further comprising administering to an individual as test AAV composition determined to have low immunostimulatory potential.

25. A kit for use in the methods of claim 1.
